(19) United States
(12) Patent Application Publication
(10) Pub. No.: US 2008/0274067 A1
(43) Pub. Date: Nov. 6, 2008

(54) Tooth Whitening Composition

(75) Inventors: Rachel Sarah Chaffer, Surrey (GB); Emma Jane Dewstowe, Surrey (GB); Robert John Gambogi, Parsippany, NJ (US); Jennifer Jane Gordon, Surrey (GB); Evelyn Elizabeth Newby, Surrey (GB); Jose Luis Velada, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, GREENFORD, MIDDESEX UNITED KINGDOM (GB)

(21) Appl. No.: 11/571,052

(22) PCT Filed: Jun. 29, 2005
(86) PCT No.: PCT/EP05/07137

§ 371 Date: Mar. 3, 2008

(30) Foreign Application Priority Data
Jul. 1, 2004 (GB) 041483.7

(51) Int. Cl. A61K 8/22; A61Q 11/00

(52) U.S. Cl. 424/53; 424/49

(57) ABSTRACT

An oral care composition comprising an active agent e.g. a whitening agent and a carrier including a carrier base comprising a wax and a substantivity agent. A method of whitening the teeth using the composition is also disclosed.

The present invention relates to a composition for use in oral care. More specifically the invention relates to a composition comprising an active agent(s) e.g. a tooth whitening agent, and a carrier, wherein the carrier includes a delivery system for the active agent(s).

Active agents or principles are commonly utilized in oral care products e.g. for the treatment of disease such as caries, tooth sensitivity, erosion or gingivitis, or to modify aesthetic properties within the oral cavity e.g. tooth whitening and oral malodor. Delivery of active agents to a site of action within the oral cavity e.g. a tooth surface, may be a requirement for obtaining an efficacious response. Another important factor may include the exposure or contact time of an active agent with a surface to be treated, in particular if slow or extended release of an active agent is desired. If an active agent does not retain contact with a surface for a sufficiently long period of time, then efficacy may not be maximized or even achieved at all. In some cases efficacy may be enhanced by mixing the active agent or principle with an accelerating or activating agent at point of use.

One area of oral healthcare that utilizes different types of carrier systems is that of tooth whitening. White teeth have long been considered cosmetically desirable. Unfortunately, teeth almost invariably become discoloured in the absence of intervention. Several factors contribute to tooth or enamel discoloration, but the three main factors are believed to be: (i) formation of plaque and tartar matrices on the tooth surface which then entraps stains resulting in natural stain that accumulates on teeth; (ii) ingestion of certain drugs during gestational tooth formation; and (iii) discoloration due to oral cavity traumatization following which blood break-down products seep into the mineralized area of the teeth during enamel formation.

There are various approaches to enamel whitening currently in general use. One approach, namely a chemical process, involves the use of a tooth whitening or bleaching formulation applied to a stained tooth surface for a specified period, after which the formulation is removed. Oxidizing agents represent one of the most widely distributed and utilized active agents in commercially available tooth whitening or bleaching products. Peroxide-containing agents such as carbamide peroxide, hydrogen peroxide and calcium peroxide are the most commonly used oxidizing agents, and are typically formulated into a liquid, solution, gel or paste. Products containing such agents may lose their whitening efficacy with time. Aqueous peroxide formulations may have only a brief period of efficacy when applied to teeth in the oral cavity because of rapid decomposition of peroxide on exposure to the enzyme, catalase, present in high concentrations in saliva. Further, as a result of the constant flushing effects of salivary secretions, the peroxide whitening agent may not remain in contact with teeth for the necessary time period to effect substantive whitening. It is known that occasional applications of a whitening agent by the dentist or by the patient himself, and even the occasional use of a tooth whitening paste or gel may not be sufficient to provide a noted improvement.

Over-the-counter teeth whitening preparations have been developed to address the cosmetic preference of many to restore luster to tooth enamel discolored by surface entrapped materials. While all dentifrices and mouthwashes contain some cleaning and polishing agents, some enamel deposits become intractable to being fully removed by these agents under normal use conditions. For example, smokers often develop discolored enamel because the tars and particulate in exhaled cigarette smoke collect on the teeth. Further, a number of comestibles, such as tea, or some medicinal agents, can stain or discolor tooth enamel.

WO96/15769 (Goulet) discloses the concept of using a chewing gum or wax base mixed with a tooth whitening agent as a means of whitening teeth. However there are no formulation or composition details provided.

WO04/108004 (Brown et al) discloses a whitening compound comprising a carrier and a whitening compound that includes an active whitening agent which is activatable by water from a tooth to which the whitening compound is applied. One method of whitening the teeth involves painting a solid or semi-solid viscous whitening compound directly onto the user's teeth.

More recently strip-type materials, known as whitening strips, have been developed and commercialized for the purpose of whitening teeth. However these strips also suffer from drawbacks such as not being sufficiently flexible and pliable enough to contact all desired surfaces of teeth in the oral cavity and thus not providing uniform whitening across the teeth surfaces.

There remains a need for oral care products that are capable of effectively delivering an agent e.g. to a surface within the oral cavity and that can adhere to the surface for a period of time to enable an efficacious response to be achieved, and where necessary to keep incompatible actives separate until point of use. For example there is a need for products that can enhance the appearance of teeth by whitening all desired surfaces of the teeth, and wherein the disadvantages and drawbacks referred to herein are minimized or eliminated altogether. Accordingly one aspect of the invention is concerned with treatment of tooth discoloration.

It is an object of the present invention to provide an oral care composition comprising a tooth whitening agent and a carrier wherein the carrier includes a delivery system capable of both delivering the tooth whitening agent to a tooth surface and of retaining the agent on the tooth surface for a sufficient period of time to enable the whitening agent to achieve a tooth whitening effect.

In a first aspect of the invention there is provided an oral care composition comprising an active agent e.g. a whitening agent, and a carrier including a carrier base comprising a wax and a substantivity agent.

Suitably the active agent of any composition according to the invention includes a whitening agent, although other actives or agents may also be used, optionally in combination with a whitening agent. Preferably whitening agents that are not used in a solubilized form are ground or manufactured to minimize particle size e.g. by micronization. Advantageously smaller particles enhance the rate of dissolution on contact with an aqueous environment, e.g. on application to the oral cavity.

Suitably the whitening agent includes a peroxide whitening compound e.g. hydrogen peroxide, carbamide peroxide, calcium peroxide, persulfate salts, percarbonate salts, perborate salts, PVP-peroxide complexes, entrapped or encapsulated forms of peroxide, STP, metal chlorites such as calcium chlorite, barium chlorite, magnesium chlorite, sodium chlorite and potassium chlorite; peroxyacids, persulfates, chlorine dioxide, other hydrogen peroxide complexes and mixtures thereof.

Suitably the whitening agent is used in an amount ranging from 0.1 to 35% w/w of the composition, preferably 2 to 15% w/w based on the hydrogen peroxide or the proportional amount of active oxygen based on the whitening moiety.

A composition according to the invention is capable of adhering to natural teeth and comprises a “substantivity agent”. By the term “substantivity agent” is meant an agent or material that is capable of adhering the composition to a surface within the oral cavity, e.g. a tooth surface, for a sufficient time period to enable an efficacious response to be achieved. Suitably when the composition comprises a tooth whitening agent, the composition adheres to a surface of the tooth or teeth to be treated i.e. whitened, typically the outward facing surface of the front teeth. The longer the contact time with a tooth surface, the greater the potential for an active ingredient(s) to penetrate into the tooth structure, thus improving efficacy. Suitable substantivity agents for use in the present invention include gums e.g. xanthan, tragacanth, carrageenan, arabic, guar, ghatti; alginates, polyvinyl alcohol and its copolymers and derivatives; carboxymethyl cellulose, ethylcellulose, hydroxyalkyl cellulose comprises hydroxyethyl cellulose, hydroxypropyl cellulose, hydroxybutyl cellulose, hydroxypropylmethyl cellulose, cellulose acetate, chitin, chitosan and their derivatives; polyacrylates such as polyacrylic acid, poly(hydroxy ethyl methacrylate), methylmethacrylate copolymer, carboxyvinylcopolymer, polyacrylamides; poly(methylvinylether-co-maleic anhydride) and its derivatives; poly(vinylpyrrolidone) its copolymers and derivatives; polyethylene oxide, polypropylene oxide, block copolymers of ethylene and propylene oxide; polybutylene and its derivatives and combinations thereof. A preferred substantivity agent is xanthan gum.

Suitably the substantivity agent is used in an amount ranging from 0.5% to 10% w/w of the composition, preferably from 1% to 5% w/w of the composition.

The compositions of the invention may comprise an active releasing agent to improve release of the agent on hydration in the oral cavity. Although not bound by any particular mechanism of action, the inventors believe that such an agent provides hydration channels in the composition milieu, allowing water to penetrate the composition and allowing the active component to leach out. When a peroxide whitening agent is used the composition comprises a peroxide releasing agent. Suitable peroxide releasing agents include water-soluble/swellable polymers e.g. xanthan gum, polyvinyl pyrrolidone, polyacrylic acid, maleic acid copolymers, polyvinyl alcohol, PEG, PPG, copolymers of PEG and/or PPG, and other suitable polymers containing either repetitive alcohol or organic acid functionalities. Xanthan gum may perform a dual role in a composition according to the invention, acting as both a substantivity agent and as a peroxide releasing agent.

Suitably the peroxide releasing agent is used in an amount ranging from 0.5 to 20-% w/w of the composition, preferably from 2 to 10% w/w of the composition. The peroxide releasing agent may be utilized to control the release rate of peroxide e.g. more releasing agent for faster release and less for a slow release.

Accelerating agents facilitate the breakdown and/or activation of water-soluble whitening agents of anhydrous compositions, on exposure to a hydrophilic medium, as may be found in the oral cavity. Hitherto use of accelerating agents in a single composition gel-based system has been generally avoided for stability reasons, as such agents can adversely affect long-term storage stability of the whitening agents. When their use has been required, it has been necessary to keep the accelerating and whitening agents apart, until just prior to activation. This has resulted in the use of costly multichambered devices and two-part formulations.

Suitable accelerating agents for use in the present invention include pH adjustors e.g. STP, sodium carbonate, sodium bicarbonate, buffers (eg. at pH 10), organic or inorganic salts of transition metals, eg. ammomium molybdate and metal ions e.g. an iron catalyst, organic and inorganic acids and bases. STP may perform a dual action, acting as both a whitening agent and an accelerating agent for other peroxide whitening components present in a composition of the invention. Preferably accelerants that are not used in a solubilized form are ground or manufactured to minimize particle size e.g by micronization. Suitably the accelerating agent is used in an amount ranging from 0.5 to 20% w/w of the composition, preferably from 1 to 15% w/w of the composition.

The active agent and/or the accelerant may be suitably encapsulated to provide additional protection to the individual ingredients. This additional protection has a favourable affect on the long-term stability. Encapsulation using waxes or polymers may be achieved by standard techniques such as spray drying and coacervation. A suitable encapsulated hydrogen peroxide is available from Amcol Health & Beauty Solutions, Inc, 1500 West Shure Drive, Arlington Heights, Ill., 60004 USA (Poly-Pore 337HP). Examples of other suitable encapsulated materials can be obtained from Tagra Biotechnologies Ltd, 8 Hemlacha Street, P.O. Box 8213, Netanya 42293, Israel, such as menthol (Tagrol Ment 1) and Vitamin E (Tagravit E1).

Suitably an oral care composition of the invention may be formulated as a stick type product. Such a product may comprise a bar of an apparently firm solid material held within a dispensing container which when applied to a surface to be treated, retains its structural integrity and shape. When a portion of the stick is drawn across a surface, a film of the stick composition is transferred to the surface. Suitably a composition of the invention may exist in the form of a firm stick e.g. resembling a lip balm or in the form of a soft stick e.g. resembling a lipstick.

Whilst the composition of the invention is essentially both hydrophobic and anhydrous, the carrier for the composition is essentially non-aqueous. Although free water is not added to the composition, it will be understood that small amounts of water, typically 5-10% w/w, may be included as a result of being present in one or more component parts prior to formulation in the composition. For example when using hydrogen peroxide as a whitening agent, formulated as a “stock” 30% w/w hydrogen peroxide solution.

A composition of the invention contains a carrier including a carrier base comprising a wax in which an active agent is incorporated. As used herein, the term “wax” includes wax-like materials which exhibit wax-like properties e.g. a material that when applied to a surface retains structural integrity and shape. Suitable waxes for use in the invention include natural, vegetable and petroleum waxes e.g. soybean wax, carnuba wax, beeswax, white beeswax, paraffin waxes such as white soft paraffin, palm wax, candelilla wax, castor wax, ceresine wax, ozokerite wax, microcrystalline wax, butyrospermum parkii, spermaceti, silicone wax, lanolin, hard butters containing high levels of lauric triglycerides (lipids), hemi synthetic glycerides consisting of satuarated fatty acids from C₈ to C₁₋₈ trylgycerides, higher alcohols such as octyldodecanol, and derivatives and mixtures of the abovementioned waxes.

The preferred carrier base is a lipid e.g Gelucire 33/01. The benefits of this composition are that it has good aesthetics, it is easy to manufacture and does not require high processing temperatures. In fact the manufacturing process can be achieved at temperatures less than 50° C. This is particularly suitable for heat sensitive materials such as peroxides. Gelucire 33/01 is available from Gattefosse UK Ltd, Arc House, Terrace Road South, Binfield, Bracknell, Berkshire, U.K., RG42 4P2.

Suitably a composition of the invention includes a carrier base comprising a wax and an oil as a softening agent to enhance softness of the composition. As used herein the term “oil” includes oily substances. Suitable oils include mineral oil, silicone oil, fatty acid ester oils e.g. triglycerol esters, preferably containing saturated alkane carboxylic acids e.g. fatty acid triglycerides selected from synthetic or naturally occurring oils e.g. olive oil, sunflower oil, soybean oil, peanut oil, rape seed oil, almond oil, palm oil, coconut oil, castor oil, wheat germ oil, grape seed oil, thistle oil, evening primrose oil, macadamia oil and the like. Preferably any unsaturated naturally occurring oil that is hydrogenated. Other fatty acid ester oils may be used wherein the acid ester contains a carboxylic acid that is either straight chained or branched, preferably saturated, and having a chain length of 3 to 30 carbon atoms. The alcohol component from the fatty acid ester will be branched and/or unbranched, preferable saturated, and will consist of 3 to 30 carbon atoms. Suitably fatty acid ester oils may be chosen from the group consisting of isopropylmyristate, isopropyl stearate, isopropyl palmitate, isopropyloleate, n-butyl stearate, n-hexyl laurate, n-decyl oleate, iso octyl stearate, isononylstearate, isononyl isononanoate, 2-ethylhexyl palmitate, 2-ethylhexyl laurate, 2-hexyldecyl stearate, 2-octyldodecyl palitate, oleyl oleate, oleyl erucate, erucyl oleate, erucyl erucate and synthetic and natural mixtures of such esters.

The amount of wax and oil (if present), as well as the relative proportion of wax to oil, may vary, according to the form of the composition. Suitably a composition of the invention, e.g. in the form of a wax stick, may contain a carrier base comprising a wax, absent of any oil. Suitably a composition of the invention, e.g. in the form of a wax stick, may contain a carrier base comprising at least 60% by weight of the composition, preferably at least 70% by weight of the composition. Suitably a soft stick composition, e.g. resembling a lipstick, may comprise a carrier base including a wax and an oil comprising at least 60% by weight of the composition, preferably at least 70% by weight of the composition, and a wax to oil ratio in the range of from about 20-40 part wax to about 80-60 part oil. Typically a firm stick composition, e.g. resembling a lip balm, may comprise a carrier base including a wax and an oil comprising at least 60% by weight of the composition, preferably at least 70% by weight of the composition, and a wax to oil ratio in the range of from about 50-90 part wax to about 50-10 part oil.

A composition according to the invention may comprise for example a single phase or a two phase carrier system e.g. in the form of an emulsion. In a second aspect there is provided an oral care composition in the form of an emulsion comprising an active agent, e.g. a whitening agent, an active release agent and a carrier including a carrier base comprising a wax and an oil as a first phase, and a second phase immiscible with the first phase, and wherein the carrier further comprises a substantivity agent and an emulsifying agent. An emulsion according to the invention may comprise substantial viscosity, even to the extent of being able to sustain its own shape for a time.

A composition of the invention in the form of an emulsion will contain a wax and an oil as hereinabove described. Suitably an emulsion will contain a carrier base comprising at least 30% by weight of the composition, preferably at least 40% by weight of the composition. Suitably an emulsion will comprise a wax to oil ratio in the range of from about 20-30 part wax to about 80-70 part oil.

Suitably when the composition is in the form of an emulsion and the carrier base comprises a first and a second phase, the first phase is essentially immiscible with the second phase. Typically the second phase may comprise one or more of the following solvents: glycerin, propylene glycol, a low molecular weight polyethylene glycol and mixtures thereof. Suitably the second phase may comprise up to 30% by weight of the composition, preferably up to 20% by weight of the composition.

When a composition of the invention is in the form of an emulsion, suitably the composition will comprise an emulsifying agent. Suitably an emulsifying agent will have an HLB value in the range 9.3 to 9.6. Exemplary emulsifying agents include polyoxyethylene alkyl ethers, polysorbate and sorbitan esters, sucrose esters and combinations of glyceryl monostearate and polyoxyethylene stearate. Suitably the emulsifying agent is used in an amount ranging from 0.1 to 10% w/w of the composition, preferably from 0.5 to 5% w/w of the composition.

As apparent hereinabove, whilst the present invention is focused on tooth whitening, it will be understood that other actives or agents may be used either in combination with a whitening agent(s) or in the absence of a whitening agent with a carrier according the invention. Suitable additional or alternative actives and agents include antimicrobial compounds e.g. triclosan, zinc salts, stannous fluoride, chlorhexidine, hexetidine, sanguinarine, benzalkonium chloride, salicylanilide, domiphen bromide, cetylpyridinium chloride, tetradecylpyridinium chloride (TPC), N-tetradecyl-4-ethylpyridinium chloride (TDEPC), octenidine, delmopinol, octapinol and other piperidino derivatives, nicin, zinc/stannous ion agents, antibiotics such as augmentin, amoxicillin, tetracycline, doxycycline, minocycline and metronidazole, essential oils such as thymol, geraniol, carvacrol, citral, hinokitiol, eucalyptol, catechol, methyl salicylate, hydrogen peroxide, metal salts of chlorite and mixtures of all the above; mineralization compounds such as sodium monofluorophosphate, water-soluble salts of fluoride e.g. sodium fluoride, potassium fluoride and stannous fluoride, sodium fluorosilicate and ammonium fluorosilicate; mineralization combinations e.g. calcium salt/phosphate salt, calcium salt/ionic fluoride sources, zinc salt/phosphate salt; desensitization compounds e.g. water-soluble potassium salt e.g. potassium nitrate, potassium citrate, potassium chloride, potassium bicarbonate and potassium oxalate; anti-calculus agents such as phosphates, pyrophosphate, phosphonates such as ethane-1-hydroxy-1,1 diphosphonate, 1-azoacycloheptane-1,1-diphosphonate, polyphosphonates, polyacrylates and other polycarboxylates, ethylenediaminetetraacetic acid and other calcium chelators, carboxylic acids and their salts and zinc salts e.g. sodium zinc citrate; anti-inflammatory agents such as NSAIDs such as ketorolac, flurbiprofen, ibuprofen, naproxen, indomethacin, aspirin, ketoprofen, piroxicam and meclofenamic acid; antioxidants such as Vitamin E, ascorbic acid, uric acid, carotenoids, Vitamin A, flavenoids and polyphenols, herbal antioxidants, melatonin, aminoindoles and lioic acids.

Additionally, compositions of the present invention will suitably contain pharmaceutically and cosmetically acceptable additives or excipients conventional in the field of oral care including flavouring agents e.g. flavouring oils e.g. oils of spearmint, peppermint, wintergreen, sassafras, clove, sage eucalyptus, marjoram, propenyl guaethol and methyl salicylate; sweetening agents including sucrose, lactose, maltose, sorbitol, xylitol sodium cyclamate and sodium saccharin; effervescing agents, and preservatives. The additives or excipients used in any given composition will be compatible both with each other and with the essential ingredients of the composition such that there is no interaction which would impair the performance of the active ingredients. All additives or excipients must of course be non-toxic and of sufficient purity to render them suitable for human use.

The compositions of the invention may be prepared by methods well known in the art and readily available to the skilled formulator. The present invention extends to a process for preparing an oral care composition as hereinbefore defined generally as comprising admixture of the component(s) of the composition. A wax stick type composition according to the present invention may be prepared by heating a wax to a suitable temperature e.g. at least to 35° C., then adding other components of the composition e.g. a whitening agent and one or more substantivity agents, and then cooling the mixture formed to ambient temperature. Alternatively a lipstick or lip balm type composition according to the invention may be prepared by heating the carrier base components e.g. the wax and oil components to a suitable temperature e.g. a temperature of at least 75° C., preferably in the range 75° C. to 85° C. and then cooling to ambient temperature. Other formulation ingredients including the active agent and the substantivity agent are then added before the composition solidifies. Suitably when preparing an emulsion type product comprising two phases, i.e. a carrier base comprising wax and an oil as a first phase and a second phase comprising components immiscible with a first phase, are heated separately to a temperature of at least 75° C., preferably in the range 75° C. to 85° C., and then mixed. Other components of the product including the active agent, the substantivity agent and the emulsifying agent are added after an emulsion has been formed and the composition has been cooled, suitably to a temperature of 30 to 40° C.

The present invention additionally encompasses the use of an oral care composition as hereinbefore defined as a cosmetic treatment for improving the appearance of teeth.

The present invention also provides a method of whitening teeth i.e. a method of improving the whiteness of teeth. The method has the step of applying onto the teeth a composition as hereinbefore defined, for a period of time sufficient to produce in the course of treatment, as known in the art, improvement in the whiteness of the teeth.

The following examples further describe and demonstrate compositions falling within the scope of the invention. For the avoidance of doubt, the examples are solely for the purpose of illustration and are not limiting with respect to the scope of the invention.

### EXAMPLES

### Example 1

### Single Phase

### Lipstick Type Formulation

| | % |
|---|---|
| Microcrystalline wax | 7.00 |
| Beeswax (white) | 14.00 |
| Mineral oil | 49.00 |
| Carbamide peroxide | 10.00 |
| Sodium carbonate | 1.00 |
| Xanthan | 9.00 |
| STP | 5.00 |
| PVP | 5.00 |
| Total | 100.00 |

Heat microcrystalline wax, beeswax and half the mineral oil to about 80° C., ensuring waxes are completely melted and homogeneous. Cool slightly then add the PVP. Allow mixture to cool to about 40° C. then add the peroxide. Slurry the carbonate, STP and xanthan in the remaining mineral oil and then mix into the batch.

### Example 2

### Single Phase

### Lip Balm Type Formulation

| | % |
|---|---|
| Microcrystalline wax | 19.00 |
| Beeswax (white) | 37.00 |
| Mineral oil | 14.00 |
| Carbamide peroxide | 10.00 |
| Sodium carbonate | 1.00 |
| Xanthan | 9.00 |
| STP | 5.00 |
| PVP | 5.00 |
| Total | 100.00 |

Heat microcrystalline wax, beeswax and half the mineral oil to about 80° C., ensuring waxes are completely melted and homogeneous. Cool slightly then add the PVP. Allow mixture to cool to about 40° C. then add the peroxide. Slurry the carbonate, STP and xanthan in the remaining mineral oil and then mix into the batch.

### Example 3

### Two Phase Emulsion

### Lipstick Type Formulation

| | % |
|---|---|
| (A) Microcrystalline wax | 4.00 |
| (A) Beeswax (white) | 8.00 |
| (A) Mineral oil | 36.00 |
| (A) Sorbitan stearate | 4.95 |
| (B) Propylene glycol | 15.00 |
| (B) Tween 20 | 0.05 |
| PVP | 5.00 |
| Sodium carbonate | 1.00 |
| STP | 5.00 |
| Xanthan | 9.00 |
| Hydrogen peroxide | 12.00 |
| Total | 100.00 |

Separately heat phase A and phase B to about 80° C. Add phase B to phase A with high shear mixing for 2 minutes, reduce speed and continue to homogenise for a further 2 minutes. Continue mixing with a paddle stirrer until cool; about 35° C. Then stir in the peroxide, PVP, carbonate, STP and xanthan.

### Example 4

### Two Phase Emulsion

### Lipstick Type Formulation

| | % |
|---|---|
| (A) Microcrystalline wax | 4.00 |
| (A) Beeswax (white) | 8.00 |
| (A) Mineral oil | 36.00 |
| (A) Sorbitan stearate | 1.25 |
| (B) Propylene glycol | 15.00 |
| (B) Arlacel 165 | 3.75 |
| PVP | 5.00 |
| Sodium carbonate | 1.00 |
| STP | 5.00 |
| Xanthan | 9.00 |
| Hydrogen peroxide | 12.00 |
| Total | 100.00 |

Separately heat phase A and phase B to about 80° C. Add phase A to phase B with high shear mixing for 2 minutes, reduce speed and continue to homogenise for a further 2 minutes. Continue mixing with a paddle stirrer until cool; about 35° C. Then stir in the peroxide, PVP, carbonate, STP and xanthan.

### Example 5

### Two Phase

### Emulsion

### Lipstick Type Formulation

| | % |
|---|---|
| (A) Microcrystalline wax | 4.00 |
| (A) Beeswax (white) | 8.00 |
| (A) Mineral oil | 36.00 |
| (A) Sorbitan stearate | 4.95 |
| (B) Propylene glycol | 17.00 |
| (B) Tween 20 | 0.05 |
| PVP | 5.00 |
| Sodium carbonate | 1.00 |
| STP | 5.00 |
| Xanthan | 9.00 |
| Carbamide peroxide | 10.00 |
| Total | 100.00 |

Separately heat phase A and phase B to about 80° C. Add phase B to phase A with high shear mixing for 2 minutes, reduce speed and continue to homogenise for a further 2 minutes. Continue mixing with a paddle stirrer until cool; about 35° C. Then stir in the PVP, peroxide, carbonate, STP and xanthan.

### Example 6

### Two Phase

### Emulsion

### Lipstick Type Formulation

| | % |
|---|---|
| (A) Microcrystalline wax | 4.00 |
| (A) Beeswax (white) | 8.00 |
| (A) Mineral oil | 36.00 |
| (A) Sorbitan stearate | 1.25 |
| (B) Propylene glycol | 17.00 |
| (B) Arlacel 165 | 3.75 |
| PVP | 5.00 |
| Sodium carbonate | 1.00 |
| STP | 5.00 |
| Xanthan | 9.00 |
| Carbamide peroxide | 10.00 |
| Total | 100.00 |

Separately heat phase A and phase B to about 80° C. Add phase A to phase B with high shear mixing for 2 minutes, reduce speed and continue to homogenise for a further 2 minutes. Continue mixing with a paddle stirrer until cool; about 35° C. Then stir in the peroxide, PVP, carbonate, STP and xanthan.

### Example 7

### Single Phase

### Wax Stick Formulation

| | % |
|---|---|
| Gelucire 33/01 | 77.00 |
| Xanthan | 2.00 |
| PVP K90 | 5.00 |
| Carbamide peroxide | 16.00 |
| Total | 100.00 |

Warm Gelucire to about 35° C. or until melted. Gently stir in the PVP, peroxide and xanthan. Continue stirring until cool and solidified.

### Example 8

### Single Phase

### Wax Stick Formulation

| | % |
|---|---|
| White soft paraffin | 77.00 |
| Xanthan | 2.00 |
| PVP K90 | 5.00 |
| Carbamide peroxide | 16.00 |
| Total | 100.00 |

Warm WSP to about 55° C. or until melted. Gently stir in the PVP, peroxide and xanthan. Continue stirring until cool and solidified.

### Example 9

### Single Phase

### Wax Stick Formulation

| | % |
|---|---|
| Gelucire 39/01 | 77.00 |
| Xanthan | 2.00 |
| PVP K90 | 5.00 |
| Carbamide peroxide | 16.00 |
| Total | 100.00 |

Warm Gelucire to about 35° C. or until melted. Gently stir in the PVP, peroxide and xanthan. Continue stirring until cool and solidified.

### Example 10

### Single Phase

Wax Stick Formulation with (A) and without (B) Accelerating Agent

| | A | |
|---|---|---|
| | % | B |
| Gelucire 33/01 | 66.00 | 71.00 |
| Xanthan | 2.00 | 2.00 |
| Peroxydone XL 10 | 27.00 | 27.00 |
| STP | 5.00 | |
| Total | 100.00 | 100.00 |

Warm Gelucire to about 35° C. or until melted. Gently stir in the Peroxydone and xanthan. Continue stirring until cool and solidified.

### Example 11

### Single Phase

### Wax Stick Formulation

| | % |
|---|---|
| White soft paraffin | 71.00 |
| Xanthan | 2.00 |
| Peroxydone XL 10 | 27.00 |
| Total | 100.00 |

Warm WSP to about 55° C. or until melted. Gently stir in the Peroxydone and xanthan. Continue stirring until cool and solidified.

Two test formulations were prepared: formulation A and comparative formulation B. The addition of a water soluble/swellable polymer such as xanthan, improved peroxide release, as shown in the examples above.

### Example 12

### Single Phase

Lipid Stick Formulation with Flavour

| | % w/w |
|---|---|
| Gelucire 33/01 | 54.70 |
| Xanthan | 2.00 |
| Peroxydone K90 | 27.00 |
| Glycerin | 15.00 |
| Sodium saccharin | 0.30 |
| Flavour | 1.00 |
| Total | 100.00 |

Dissolve saccharin in warm glycerin. Warm Gelucire to about 35° C. or until melted. Gently stir in the warm glycerine/saccharin mixture, flavour, xanthan and Peroxydone. Continue stirring until cool and solidified.

### Example 13

### Single Phase

Lipid Stick Formulation with Flavour and Accelerating Agent.

| | % w/w |
|---|---|
| Gelucire 33/01 | 49.70 |
| Xanthan | 2.00 |
| Peroxydone K90 | 27.00 |
| STP | 5.00 |
| Glycerin | 15.00 |
| Sodium saccharin | 0.30 |
| Flavour | 1.00 |
| Total | 100.00 |

Dissolve saccharin in warm glycerin. Warm Gelucire to about 35° C. or until melted. Gently stir in the warm glycerine/saccharin mixture, flavour, STP, xanthan and Peroxydone. Continue stirring until cool and solidified.

### Example 14

### Single Phase

Lipid Stick Formulation with Flavour and Encapsulated Accelerating Agent.

| | % w/w |
|---|---|
| Gelucire 33/01 | 52.00 |
| Xanthan | 2.00 |
| Poly-Pore 337HP | 27.00 |
| Encapsulated sodium carbonate | 2.70 |
| Glycerin | 15.00 |
| Sodium saccharin | 0.30 |
| Flavour | 1.00 |
| Total | 100.00 |

### Determination of Gel Substantivity

### Method

Hydrated bovine teeth were treated with gel that was labelled with a dye e.g. erythrosin. The treated teeth were then placed in a shaker bath containing Phosphate Buffer Saline. The treated bovine teeth were then assessed for the percentage of gel remaining every 5 minutes for 30 minutes. The Substantivity Score was then measured as the percentage of gel remaining through the course of the experiment.

### Results

| Time (mins) | Example 10 A | Example 10 B |
|---|---|---|
| 5 | 1 | 1 |
| 10 | 1 | 1 |
| 15 | 1 | 1 |
| 20 | 1 | 1 |
| 25 | 1 | 1 |
| 30 | 1 | 1 |

### Where: —

| | |
|---|---|
| 1 | >90% left on the tooth |
| 2 | >50% left on the tooth |
| 3 | >30% left on the tooth |
| 4 | <10% left on the tooth |

### Conclusion

The results indicate that the formulation is substantive for at least 30 minutes.

### Determination of Bleaching Effects Using Bovine Enamel Whitening Model.

The bleaching effects of the composition disclosed in Example 10 above was determined and compared to the bleaching effects observed with a commercially available preparation.

### Method

Bovine teeth were used and L* (from CIE 1976 L*a*b* colour space scale) was measured at the start of the experiment using a spectrocolorimeter. Example 10 was applied to the teeth and these were placed into a container such that a liquid substantivity challenge was applied to the treated teeth. At the end of the treatment time the teeth were rinsed and dried and L* was remeasured using a spectrocolorimeter. This method was repeated for a number of days to mimic in-vivo night time use of the product. At the end of the experiment the overall change in L* (i.e ΔL) was calculated.

Results

### CONCLUSION

The results indicate that the change in L (ΔL) after 12 hours treatment was higher than a commercially available night time product. The results are illustrated graphically above.

1. An oral care composition comprising an active agent e.g. a whitening agent, and a carrier including a carrier base comprising a wax and a substantivity agent.

2. An oral care composition according to claim 1, wherein the wax is a lipid.

3. An oral care composition according to claim 2 further comprising a softening agent e.g. an oil.

4. An oral care composition comprising an active agent e.g. a whitening agent, and a carrier including a carrier base comprising a wax and an oil as a first phase and a second phase immiscible with the first phase and wherein the carrier further comprises a substantivity agent and an emulsifying agent.

5. An oral care composition according to claim 1 wherein the active agent comprises a whitening agent.

6. An oral care composition according to claim 1 wherein the active agent comprises an antimicrobial compound, mineralization compound, desensitization compound, anti-calculus agents, anti-inflamatory agents or anti-oxidants.

7. An oral care composition according to claim 5 wherein the whitening agent comprises a peroxide whitening agent and the composition further comprises a peroxide releasing agent.

8. An oral care composition according to claim 1 further comprising an accelerating agent.

9. (canceled)

10. A method of whitening teeth comprising applying onto teeth a composition according to claim 1, for a period of time sufficient to produce an improvement in the whiteness of the teeth.

11. A method of preparing a composition according to claim 1 which method comprises an admixture of components of the composition.
